(19) United States
(12) Patent Application Publication
(10) Pub. No.: US 2010/0047343 A1
(43) Pub. Date: Feb. 25, 2010

(54) MULTIPARTICULATE FORMULATION HAVING TRAMADOL IN IMMEDIATE AND CONTROLLED RELEASE FORM

(75) Inventors: John L. Haslam, Lawrence, KS (US); Roger A. Rajewski, Lawrence, KS (US)

Correspondence Address: INNOVAR, LLC, P O BOX 250647, PLANO, TX, 75025, US

(73) Assignee: The University of Kansas, Lawrence, KS (US)

(21) Appl. No.: 12/608,686

(22) Filed: Oct. 29, 2009

Related U.S. Application Data
(63) Continuation of application No. PCT/US07/71228, filed on Jun. 14, 2007.
(60) Provisional application No. 60/830,368, filed on Jul. 12, 2006.

(51) Int. Cl. A61K 9/58; A61K 31/135; A61K 9/16

(52) U.S. Cl. 424/462; 514/646; 424/497

(57) ABSTRACT

A multi-particulate pharmaceutical composition of rapid release particles and controlled release particles comprising tramadol or a salt thereof is provided. The composition provides a rapid and controlled release of tramadol or a salt thereof in a substantially pH independent manner after oral administration to a subject. The composition can be included in a capsule, caplet, sachet, or other solid dosage form adapted to retain and then release solid pharmaceutical compositions.

### CROSS-REFERENCE TO EARLIER FILED APPLICATIONS

The present invention application is a continuation of and claims the benefit of PCT International Application No. PCT/US2007/071228, filed 14 Jun. 2007, which claims the benefit of U.S. Provisional Application of 60/830,368, filed 12 Jul. 2006, the entire disclosures of which are hereby incorporated by reference.

### FIELD OF THE INVENTION

The present invention concerns a multi-particulate formulation containing tramadol for the dual release of tramadol following oral administration to a subject. More particularly, the invention concerns a multi-particulate composition that provides a combined rapid and controlled release of tramadol.

### BACKGROUND OF THE INVENTION

Tramadol is a centrally acting synthetic analgesic. It is generally administered as a racemic mixture of its two enantiomers. Some of the known physical properties of tramadol HCl include: a) solubility in water: 790 mg/mL at 24° C.; b) solubility in water: 840 mg/mL at 37° C.; c) pH of a saturated solution: 5.0; d) density of saturated solution: 1.1079 at 37° C.; e) viscosity at 37° C. (sat'd. sol.): 86.5 cp; and f) mp. by DSC: 181.3° C.

The enantiomers of tramadol exhibit differing affinities for various receptors. (+/−)-Tramadol is a selective agonist of mu receptors and preferentially inhibits serotonin reuptake, whereas (−)-tramadol mainly inhibits noradrenaline reuptake. The action of these two enantiomers is both complementary and synergistic and results in the analgesic effect of (+/−)-tramadol. After oral administration of ULTRAM™, tramadol HCl demonstrates 68% bioavailability, with peak serum concentrations reached within 2 hours. The elimination kinetics can be described as 2-compartmental, with a half-life of 5.1 hours for tramadol and 9 hours for the M1 derivative after a single oral dose of 100 mg. This explains the approximately 2-fold accumulation of the parent drug and its M1 derivative that is observed during multiple dose treatment with tramadol HCl

The recommended daily dose of tramadol HCl is from 50 and 100 mg every 4 to 6 hours, with a maximum dose of 400 mg/day; the duration of the analgesic effect after a single oral dose of rapid release tramadol HCl (100 mg) is about 6 hours. Adverse effects, and nausea in particular, are dose-dependent and therefore considerably more likely to appear if the loading dose is high. The reduction of this dose during the first days of treatment is an important factor in improving tolerability. Other adverse effects are generally similar to those of opioids, although they are usually less severe, and can include respiratory depression, dysphoria and constipation. Tramadol can be administered concomitantly with other analgesics, particularly those with peripheral action, while drugs that depress CNS function may enhance the sedative effect of tramadol

Racemic tramadol is commercially available in 50 mg immediate release tablets under the trademark ULTRAM™ (Ortho-McNeil), which consists of: a) 50 mg Tramadol HCl; b) corn starch; c) microcrystalline cellulose; d) lactose; e) Mg stearate; f) sodium starch glycolate; g) HPMC; h) PEG; i) PolySorbate 80; j) titanium dioxide; and k) wax.

Extended/controlled release dosage forms containing tramadol are commercially available under the trademark RALIVIRA™ ER. Capsule formulations containing tramadol in bead, granulate, pellet, powder or multi-particulate form have been reported in the literature. Patent and scientific literature publications disclose tablet or capsule formulations that provide a controlled, extended or sustained release of tramadol and a rapid release of the same.

The absence of a food effect upon administration of a capsule containing particulate tramadol HCl in controlled release form has been reported in the literature; however, the relevant formulation was only defined as being that of SMB Technologies or Laboratories SMB SA (Brussels, Belgium). Similar results were reported for a capsule containing pelletized tramadol HCl in controlled release form (Asta Medica Group or Temmler Pharma Gmbh; Marburg, Germany).

Some of the patent and literature references suggest using multi-layered coated beads or spheres having an external immediate release layer coated over an internal sustained release core. Other references suggest using an admixture of rapid release particles and coated sustained release particles included within a capsule. Some references disclose the use of a semipermeable membrane as the release rate-controlling membrane, and some of those references suggest the use of a pore former in the membrane to convert the semipermeable membrane into a microporous coating. The vast majority of those references employ ethyl cellulose, cellulose acetate, or EUDRAGIT® brand poly(methacrylates)-co-(methyl methacrylate) copolymers, to form the release-rate controlling coating surrounding the sustained release beads.

U.S. Pat. No. 6,156,342 discloses a capsule containing two different pellets or tablets, a rapid release pellet or tablet and a controlled release pellet or tablet. The controlled release pellet includes a microporous membrane containing cellulose acetate, EUDRAGIT® S100, triacetin, PEG 400 and confectioner's sugar. The release of tramadol HCl from the controlled release pellet or tablet appears to be pH dependent, as there are measurable and significant differences in tramadol release when determined in simulated gastric fluid (SGF) versus simulated intestinal fluid (SIF).

A pH dependent controlled release of tramadol is undesirable as it results in substantial inter-patient and intra-patient variability due to lack of reproducibility in the controlled release of drug. Specifically, the rate of absorption of the drug can change at it traverses the intestines, so some patients will receive drug at a substantially faster controlled rate than other. This effect can be the observed difference in pharmacokinetics observed when the dosage form is studied under fed and fasted conditions. Typically the dosage form will remain in the stomach for a longer time in the fed state than in the fasted state, which will change the amount of drug released from the dosage form in the stomach and the intestine. The other cause for the difference in the fed and fasted pharmacokinetics can be the greater absorption of the drug high in the intestinal tract verses the lower G.I. tract. In this case a longer residence in the stomach can lead to greater absorption by exposing the upper intestinal tract to a larger amount of drug. Given the potential toxicity of tramadol when administered too rapidly in high doses, a pH dependent controlled release is very undesirable.

None of the known art discloses a multi-particulate combined rapid and controlled release dosage form for tramadol, wherein the release of tramadol from the controlled release particles is substantially pH independent. Thus, a need remains for an improved multi-particulate combined rapid and controlled release dosage form that exhibits a lack of any substantial pH dependence in the controlled release of drug thereby resulting in a more reproducible drug release profile with less inter-patient or intra-patient variability.

### SUMMARY OF THE INVENTION

The present invention seeks to overcome some or all of the disadvantages inherent in the art. The present invention provides a dual rapid and controlled release of tramadol from a capsule comprising a particulate composition comprising at least two different populations of particles. The first population of particles provides a rapid release of tramadol when administered orally or when exposed to an aqueous environment. The second population of particles provides a controlled release of tramadol when administered orally or when exposed to an aqueous environment. Release of tramadol from the controlled release particles is substantially pH independent, meaning that there is less than a 10%, less than a 5%, or less than 2.5% difference in the rate of release or in the total amount of tramadol released at a given time point when release of the tramadol from the controlled release particles is compared in simulated gastric fluid versus simulated intestinal fluid.

The multi-particulate composition and dosage form of the invention are adapted for once-a-day administration to a subject. They are suitable for the treatment of pain and other indications, disorder or symptom that is therapeutically responsive to tramadol. Tramadol (free base or salt) is indicated for the management of moderate to moderately severe pain in mammals. For example, the composition can be given to treat pain associated with tooth extraction, childbirth, arthritis, injury, or inflammation. The composition can be used in combination with other pain medications. The rapid release particles are present in an amount sufficient to provide a subject with an initial loading dose of tramadol within a period of 2 hours or less, 1 hour or less, 30 minutes or less, or about 10 min or less. In some embodiments, a 100 mg dose of tramadol HCl in the rapid release particles may provide a tramadol plasma concentration in the range of 200-600 ng/mL with a Tₘₐₓ of 1-3 or 1.9-2.3 hours. The controlled release particles are present in an amount sufficient to provide a subject with a continuous maintenance dose of tramadol for a period of not less than 12 hours, not less than 16 hours, not less than 20 hours and up to about 24 hours, or up to about 28 hours, or up to about 32 hours after administration. Plasma concentrations from the controlled release portion of the formulations can range from 100 to 500 ng/mL over an 18 hour period following administration of a dose of 150-300 mg tramadol HCl in the sustained release form.

The composition of the invention can be administered to a mammal, such as a human, to provide an advantageous pharmacokinetic profile. In some embodiments, the composition is substantially bioequivalent to a known controlled release formulation containing tramadol hydrochloride. In other embodiments, the composition provides a unique tramadol plasma profile. Some embodiments of the invention provide a drug release profile as described herein.

One aspect of the invention provides a multi-particulate dosage form containing a composition of beads/particles comprising tramadol. The unit dosage form comprises an admixture of at least two different types of beads. A first group of beads provides an immediate/rapid release of tramadol, and a second group beads provides a controlled/extended release of tramadol. The beads are optionally provided in a capsule dosage form to facilitate administration; alternatively, they can be administered to a subject in loose form or in a sachet dosage form. In some embodiments, the composition is provided in a capsule, wherein during use the capsule halves are separated and the composition is sprinkled onto a soft food such as apple sauce or into a drink.

Some embodiments of the invention provide a reduced food-effect, which is generally observed with administration of the commercial immediate release product ULTRAM™.

The controlled release particles are coated with a release-rate controlling material such as cellulose acetate butyrate or cellulose acetate propionate. The core of the controlled release particle can include or exclude a release rate-controlling material. The coating includes a pore former thereby rendering the coating microporous during use. In some exemplary embodiments, a unit dose of the controlled release particles releases about 10 to 350 mg or 50 to 350 mg or about 150 mg of tramadol hydrochloride. As a loading dose or first initial dose, the amount may be reduced to below 50 mg. The in vitro dissolution rate or total amount of tramadol released from the controlled release particles is substantially pH independent.

The rapid release particles are not coated with a release-rate controlling material. A maximum time period for release of drug from these particles is generally within two hours or less, one hour or less, 30 minutes or less, 15 min or less, or 10 min or less in vitro. In some embodiments, the total amount of tramadol released from the non coated particles is >90% by wt within about 10 minutes in vitro when determined using an assay as described herein. In some exemplary embodiments, a unit dose of these uncoated rapid release particles releases about 10-100 mg or about 50 mg of tramadol HCl.

One aspect of the invention provides a multi-particulate pharmaceutical composition comprising:
a population of rapid release particles comprising tramadol and at least one pharmaceutical excipient; and
a population of coated controlled release particles comprising a core composition and a release rate-controlling coating composition enclosing the core composition, wherein the core composition comprises tramadol and at least one pharmaceutical excipient, and the coating composition comprises a semipermeable polymer, pore former and plasticizer, and the controlled release particles provide a substantially pH independent release of tramadol.

Another aspect of the invention provides a dual release capsule dosage form comprising a capsule shell and a pharmaceutical composition as defined herein.

The amount of tramadol present in the population of controlled release particles can be greater than or less than the amount of tramadol present in the population of rapid release particles. Some embodiments provide a greater amount of tramadol present in the controlled release particles than in the rapid release particles.

The invention also provides a controlled release particle that provides a substantially pH independent release of tramadol, the particle comprising:
a core comprising tramadol and at least one excipient; and
a semipermeable coating enclosing the core, the coating comprising a film-forming semipermeable polymer, a water soluble or water erodible pore former, and a plasticizer; wherein
the pore former dissolves or erodes following exposure of the particle to an aqueous environment, thereby rendering the semipermeable membrane microporous so as to provide a pH independent release of tramadol from the core.

Some embodiments of the invention include those wherein the semipermeable polymer comprises cellulose acetate butyrate or cellulose acetate propionate. Some embodiment of the invention include a plasticizer comprising PEG having a molecular weight of 200 to 8000, triethyl citrate, tributyl citrate, diethyl phthalate, or dibutyl sebacate. Some embodiments of the invention include a pore former comprising sucrose, sorbitol or hydroxypropyl methylcellulose.

Release of tramadol from the controlled release particles typically follows a first order, pseudo-first order, zero order, pseudo-zero order, or sigmoidal release profile. In some embodiments, the release of tramadol from the sustained release particles follows an approximately first order release with an initial lag time. The rapid release particles can release drug immediately or in a period of two hours or less following exposure to aqueous environment. If the particles of the invention are enclosed within a capsule shell, initial release of tramadol from the particles will be delayed at least until such time as the shell dissolves or erodes in the aqueous environment of use sufficiently to permit contact of the particles with the aqueous environment.

The dosage form of the invention can be a capsule (hard or soft), caplet, tablet, sache, particulate admixture, or other solid dosage form known in the pharmaceutical industry as being suitable for administration of particulate composition.

The ratio of tramadol present in controlled release form versus rapid release form is regulated by controlling the total weight or volume of the respective controlled release particles and rapid release particles present in a pharmaceutical composition. In some embodiments, at least 50% by wt., or about 50% to 100% by wt., or about 65% to 85% by wt. of the tramadol is present in controlled release form and no more than about 50% by wt., or about 0% to 50% by wt., or about 15% to 35% by wt. of the tramadol is present in rapid release form. The amount of the rapid and controlled release particles in the composition can be varied over a wide range. For example, the rapid release fraction would be from 0 to 100 mg or from >0 to 100 mg of tramadol HCl, and the controlled release fraction from 0 to 400 mg or from >0 to 400 mg tramadol HCl. In some embodiments, the dosage form contains about 50 mg of immediate release and 150 mg of tramadol HCl. In some embodiments, a pharmaceutical composition containing the multi-particulate composition comprises about 25 to 100 mg, or about 50 mg of tramadol HCl in rapid release form and about 50 to 300 mg, or about 150 mg of tramadol HCl in controlled release form.

### BRIEF DESCRIPTION OF THE FIGURES

The following figures form part of the present description and describe exemplary embodiments of the claimed invention. The skilled artisan will, in light of these figures and the description herein, be able to practice the invention without undue experimentation.

FIG. 1 depicts a cross-sectional view of an exemplary rapid release particle of the invention.

FIG. 2 depicts a cross-sectional view of an exemplary controlled release particle of the invention.

FIG. 3 depicts a side elevation view of a capsule containing an admixture of rapid release and controlled release particles.

FIG. 4 depicts the release profile for rapid release particles made according to Example 1, Method A.

FIG. 5 depicts the release profile for controlled release particles comprising different amounts of coating (based upon the amount of coating solution applied). The uncoated particles were made according to Example 1, Method A and then coated as in Example 2 Method A.

FIG. 6 depicts the release profile for controlled release particles comprising different amounts of coating (based upon the amount of coating solution applied). The uncoated particles were made according to Example 1, Method B then and coated as in Example 2 Method A (Same as FIG. 5 except a different batch of particles).

FIG. 7 depicts the release profile for controlled release particles. The uncoated particles were made according to Example 1, Method B and then coated as in Example 2, Method B.

FIG. 8 depicts the release profile for controlled release particles. The uncoated particles were made according to Example 1, Method B and then coated as in Example 2, Method B with differing amounts of sucrose and triethyl citrate in the coating solution. (Curve A is 35% sucrose and 30% TEC as percent of CAB 171-15PG; Curve B is 45% sucrose and 30% TEC; Curve C is 55% sucrose and 30% TEC; Curve D is 45% sucrose and 20% TEC).

FIG. 9 depicts the release profile for the combination of immediate and the extended release beads in a capsule made according to Example 3, Method B. The coated particles are prepared as in FIG. 6 using the 4400 g coating amount. All amounts of tramadol are given as the salt form, i.e. tramadol HCl. The data in FIG. 9 are from particles prepared as given in Example 3. Method B including the weights of the immediate release and extended release beads.

FIG. 10 depicts the release profile for the extended release capsule made according to Example 3, Method C. The particles were prepared as for FIG. 7. All amounts of tramadol are given as the salt form, i.e. tramadol HCl. The data in FIG. 10 are from particles prepared as in Example 3, Method C.

FIG. 11 depicts the combination in vitro release profile provided by an extended release capsule dosage form prepared according to Example 3, Method D. All amounts of tramadol are given as the salt form, i.e. tramadol HCl.

FIG. 12 depicts the in vivo plasma tramadol concentration time profile in beagles following administration of either two 50 mg of Ultram tablets (triangles) or two extended release capsule dosage forms of the present invention (squares) each consisting of 102 mg of immediate release beads containing 50 mg tramadol HCl (Example 1, Method B) and 388 mg of sustained release beads containing 150 mg tramadol HCl (immediate release beads coated as Example 2, Method B) enclosed in a #0 hard gelatin capsule.

FIG. 13 depicts the average in vivo plasma tramadol concentration time profile in either fed (closed symbols) or fasted (open symbols) beagles following administration of two extended release capsule dosage forms of the present invention each consisting of 101 mg of immediate release beads containing 50 mg tramadol HCl (Example 3, Method D) and 388 mg of sustained release beads containing 150 mg tramadol HCl (immediate release beads coated as Example 2, Method B) enclosed in #0 hard gelatin capsule.

FIG. 14 depicts the combination in vitro release profile provided by an extended release capsule dosage form prepared by mixing immediate release particles of FIG. 4 with controlled release particles of FIG. 5 (2400 g batch coating). The immediate release represent 50% and the extended release represent 50% of the total tramadol HCl

### DETAILED DESCRIPTION OF THE INVENTION

As used herein, a semipermeable polymer is a polymer or combination of polymers that forms a film used to coat the core of the controlled release particles. A semipermeable polymer permits diffusion of water into the coated controlled release particles but does not permit egress of tramadol through the coating. Instead, tramadol is released through the micropores in the coating, which micropores were formed by dissolution of the pore former initially present in the coating. Accordingly, the semipermeable membrane of the controlled release particles is converted to a microporous semipermeable membrane after exposure of the particles to an aqueous environment of use. Particularly suitable semipermeable polymers include cellulose acetate butyrate (CAB) and cellulose acetate propionate (CAP). Such materials are readily available from Eastman Chemical (Kingsport, Tenn.). CAP and CAB are available in various grades, some of which are detailed in the table below.

| | Viscosity | | | | | Melting | Tg |
|---|---|---|---|---|---|---|---|
| Type | (Poise) | Acetyl % | Butyryl % | Propionyl % | Hydroxyl % | Point ° C. | ° C. |
| CAB-551- | 0.038 | 2.0 | 53 | — | 1.5 | 127-142 | 85 |
| 0.01 | | | | | | | |
| CAB-551-0.2 | 0.76 | 2.0 | 52 | — | 1.8 | 130-140 | 101 |
| CAB-531-1 | 7.2 | 3.0 | 50 | — | 1.7 | 135-150 | 115 |
| CAB-500-5 | 19.0 | 4.0 | 51 | — | 1.0 | 165-175 | 96 |
| CAB-381-0.1 | 0.38 | 13.5 | 38 | — | 1.3 | 155-165 | 123 |
| CAB-381-0.5 | 1.9 | 13.5 | 38 | — | 1.3 | 155-165 | 130 |
| CAB-381-2 | 7.6 | 13.5 | 38 | — | 1.3 | 171-184 | 133 |
| CAB-381-20 | 76 | 13.5 | 37 | — | 1.8 | 195-205 | 141 |
| CAB-321-01 | 0.38 | 18.5 | 31.2 | — | 1.1 | 165-175 | 127 |
| CAB-171-15 | 57 | 29.5 | 17 | — | 1.1 | 230-240 | 161 |
| CAP-482-0.5 | 1.52 | 2.5 | — | 45.0 | 2.6 | 188-210 | 142 |
| CAP-482-20 | 76.0 | 2.5 | — | 46.0 | 1.8 | 188-210 | 147 |
| CAP-504-0.2 | 0.76 | 0.6 | — | 42.5 | 5.0 | 188-210 | 159 |

The information in this table was extracted from “Eastman Cellulose Esters Publication No. E-146K”, the entire disclosure of which is hereby incorporated by reference.

When used individually to form the coating, the preferred polymers, as denoted above, are CAB 381-20, CAB 171-15 and CAP-482-20. In general, a preferred semipermeable polymer will have a viscosity of about 1 to 100 poise or >50 poise and a melting point of about 100 to 300° C. or about 140 to 240° C.

The coating can also comprise a combination of semipermeable polymers. Different polymers can be combined in various proportions to achieve a desirable resultant polymer coating. The combination of different polymers will result in a polymeric mixture having a different glass transition temperature, viscosity and/or rheological properties as compared to either starting polymer. In this embodiment, higher viscosity polymers can be combined with lower viscosity polymers to form a semipermeable polymeric composition that is used to form the membrane. The exemplary polymeric mixture would posses a viscosity lower than the higher viscosity polymer and higher than the lower viscosity polymer.

As used herein, a “pore former” is a material or combination of materials that is readily soluble or erodible in an aqueous environment and that can be incorporated into the coating composition. After exposure of the controlled release particle to an aqueous environment, the pore former will dissolve or erode from the coating composition rendering the coating microporous. In other words, the micropores in the coating are formed during exposure of the controlled release particles to aqueous fluids in an intended environment of use. Tramadol will then exit the controlled release particles via the so-formed micropores.

Methods of preparing coatings wherein the micropores form in the environment of use are well known and described in, among others, U.S. Pat. No. 3,845,770, U.S. Pat. No. 3,916,899, U.S. Pat. No. 4,063,064, U.S. Pat. No. 4,088,864, U.S. Pat. No. 4,816,263, U.S. Pat. No. 4,200,098, U.S. Pat. No. 4,285,987 and U.S. Pat. No. 5,912,268, the relevant disclosures of which are hereby incorporated by reference.

Exemplary “pore formers” include poly(ethylene glycol) (PEG), carbohydrate, sugar, reduced sugar alcohol, sorbitol, polyol, xylitol, mannitol, lactose, sucrose, fructose, maltose, dextrose, water soluble or water erodible derivatized starch, water soluble or water erodible derivatized cellulose, water soluble or water erodible polymer, urea, salt, and combinations thereof. Particularly suitable materials include sugar, polyol, water soluble or water erodible derivatized cellulose, and water soluble or water erodible polymer. Specific materials include sucrose, sorbitol and hydroxypropyl methylcellulose.

For coatings prepared with non-aqueous solvents, the coating material is generally a solution rather than a suspension. To obtain a coating solution, the pore former should dissolve in the non-aqueous solvent to form the coating solution. Some of the above-mentioned pore formers mentioned may not dissolve in the unmodified non-aqueous solvent; however, one or more cosolvents can be added to the non-aqueous solvent to aid in dissolution of pore former.

The release rate of tramadol through a particular microporous coating is controlled by regulating the porosity of the coating and/or the thickness of the coating. The porosity of the coating will vary according to its composition: the greater the content of pore former in the coating, the greater porosity of the resulting microporous coating. The coating will generally comprise about 10 to 60% by wt. or 30 to 45% by wt. of pore former. The amount of pore former added to the coating may depend upon the amount of plasticizer present in the coating solution. If the plasticizer is particularly effective, it will allow the polymer in the coating solution to surround the pore former during formation of the coating thereby preventing the pore former from dissolving out when the particle is exposed to an aqueous environment. As a result, the pore former is less effective. If the plasticizer is not particularly effective, less of the pore former will be surrounded by polymer during formation of the coating and the pore former will be more effective.

Plasticizers that can be used in the coating include all those that are generally incorporated into polymeric coatings of drug delivery devices. Plasticizers generally improve the mechanical properties and increase the flexibility of the polymeric film. Plasticizers generally reduce cohesive intermolecular forces and increase mobility of polymer chains, thus reducing polymer-polymer interactions. This action is responsible for the changes to the properties of the polymers and films thereof such as a reduction of Tg (glass transition temperature) or softening temperature and the elastic module, increasing polymer flexibility, thus facilitating the process of formation of the membrane or film. A preferred pharmaceutical plasticizer is non-toxic and non-irritating; has a reduced tendency to migrate, extrude or volatilize; and has good miscibility with the polymer(s) in the film. Plasticizers that can be used in the coating include, for example and without limitation, acetyl triethyl citrate, acetyl tributyl citrate, triethyl citrate, acetylated monoglycerides, glycerol, polyethylene glycol, triacetin, propylene glycol, dibutyl phthalate, diethyl phthalate, isopropyl phthalate, dimethyl phthalate, dactyl phthalate, dibutyl sebacate, dimethyl sebacate, castor oil, glycerol monostearate, fractionated coconut oil, poly(ethylene glycol) (PEG), others or a combination thereof. In some embodiments, the plasticizer is PEG having a molecular weight of 200 to 8000, ester of citric acid, ester of phthalic acid. Specific plasticizers include PEG having a molecular weight of 200 to 8000, triethyl citrate, tributyl citrate, diethyl phthalate, and dibutyl sebacate.

Suitable plasticizers also include, by way of example and without limitation, low molecular weight polymers, oligomers, copolymers, oils, small organic molecules, low molecular weight polyols having aliphatic hydroxyls, ester-type plasticizers, glycol esters, poly(propylene glycol), multi-block polymers, single-block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. A combination of plasticizers may also be used in the present formulation. The PEG based plasticizers are commercially available or can be made by a variety of methods, such as disclosed in Poly (ethylene glycol) Chemistry: Biotechnical and Biomedical Applications (J. M. Harris, Ed.; Plenum Press, NY) the disclosure of which is hereby incorporated by reference.

A capsule according to the invention will have a storage shelf-life of no less than one week, three weeks, one month, three months, six months, one year or two years. For example, for a capsule having a shelf life of at least six months, the shell of the capsule will not fail storage stability tests for a storage period of at least six months. The criteria for acceptable shelf-life are set as needed according to a given capsule product and its storage stability requirements. It should be noted that a shelf-life of as little as one week is suitable for products that are compounded by a pharmacist and sold to customers of a pharmacy.

The term “shell” as used herein is taken to mean the shell of a capsule dosage form or the encasement or encapsulation material used to encapsulate fill compositions made from the particles. Any material suitable for use in forming a capsule shell or in encapsulating another composition can be used according to the invention.

The shell can be hard or soft and any materials suitable for preparing such shells can be used in the capsule of the invention. Materials suitable for the preparation of the capsule shell include soft gelatin, hard gelatin, hydroxypropyl methylcellulose, starch, animal gelatin, agar, fish (piscine) gelatin or a combination thereof. Other suitable materials include: polyvinyl alcohol/polyvinyl acetate copolymer (U.S. Pat. No. 3,300,546); a blend of hydroxybutyl methylcellulose and hydroxypropyl methylcellulose (U.S. Pat. No. 4,765,916); polyvinyl acetate (U.S. Pat. No. 2,560,649, U.S. Pat. No. 3,346,502); water-soluble gelatin (U.S. Pat. No. 3,525,426); polyvinyl alcohol (U.S. Pat. No. 3,528,921, U.S. Pat. No. 3,534,851, U.S. Pat. No. 3,556,765, U.S. Pat. No. 3,634,260, U.S. Pat. No. 3,671,439, U.S. Pat. No. 3,706,670, U.S. Pat. No. 3,857,195, U.S. Pat. No. 3,877,928, U.S. Pat. No. 4,367,156, U.S. Pat. No. 4,747,976, U.S. Pat. No. 5,270,054); pullulan (U.S. Pat. No. 3,784,390, U.S. Pat. No. 4,623,394, U.S. Pat. No. 6,887,307); polymers derived from such monomers as vinyl chloride, vinyl alcohol, vinyl pyrrolidone, furan, acrylonitrile, vinyl acetate, methyl acrylate, methyl methacrylate, styrene, vinyl ethyl ether, vinyl propyl ether, acrylamide, ethylene, propylene, acrylic acid, methacrylic acid, maleic anhydride, salts of any of the aforementioned acids and mixtures thereof; polyvinyl chloride; polypropylene; acrylic/maleic copolymers; sodium polyacrylate; polyvinyl pyrrolidone; glucomannan and optionally another natural polysaccharide with a polyhydric alcohol such as glycerin (U.S. Pat. No. 4,851,394); plastic and polylactide/polyglycolide (Elanco Animal Health Co.); HPMC (Shionogi Qualicaps Co. Ltd (Nara Japan); SUHEUNG CAPSULES CO. LTD. (KYUNGGI-DO, KOREA) and Capsugel); or a combination thereof. Essentially any material known to those of ordinary skill in the art as being for the preparation of capsule shell can be used in a capsule according to the invention. Suitable starch capsules can be made and used according to Vilivalam et al. (Pharmaceutical Science & Technology Today (2000), 3 (2), 64-69). A chitosan capsule for colonic delivery can be made and used according to Yamamoto (Kobunshi (1999), 48 (8), 595) or Tozaki et al. (Drug Delivery System (1997), 12 (5), 311-320). Other suitable shell materials are disclosed in U.S. Patent Application Publication No. 2002/0081331 to R. P. Scherer Technologies Inc. (Cardinal Health, Inc.), which discloses film-forming compositions comprising modified starches and iota-carrageenan.

Although not necessary, the formulation of the present invention may include a preservative, adsorbent, antioxidant, acidifying agent, alkalizing agent, antibacterial agent, antiadherent, binder, buffering agent, colorant, diluents, direct compression excipient, electrolyte, disintegrants, flavorant, glidant, opaquant, polishing agent, salt, stabilizer, sweetening agent, other excipients known by those of ordinary skill in the art for use in capsules, or a combination thereof.

As used herein, the term “alkalizing agent” is intended to mean a compound used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art.

As used herein, the term “acidifying agent” is intended to mean a compound used to provide an acidic medium. Such compounds include, by way of example and without limitation, acetic acid, amino acid, citric acid, fumaric acid and other alpha-hydroxy acids, such as hydrochloric acid, ascorbic acid, and nitric acid and others known to those of ordinary skill in the art.

As used herein, the term “adsorbent” is intended to mean an agent capable of holding other molecules onto its surface by physical or chemical (chemisorption) means. Such compounds include, by way of example and without limitation, powdered and activated charcoal and other such materials known to those of ordinary skill in the art.

As used herein, the term “antioxidant” is intended to mean an agent who inhibits oxidation and is thus used to prevent the deterioration of preparations by the oxidative process. Such compounds include, by way of example and without limitation, ascorbic acid, ascorbic palmitate, Vitamin E, Vitamin E derivative, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid or alkali. Such compounds include, by way of example and without limitation, sodium bitartrate, potassium metaphosphate, potassium phosphate, monobasic sodium acetate and sodium citrate anhydrous and dehydrate and other such materials known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose, sugar substitute, artificial sweetener, and other such materials known to those of ordinary skill in the art.

As used herein, the expression “antiadherent” is intended to mean agents that prevent the sticking of tablet formulation ingredients to the punches and dies in a machine during production. Such compounds include, by way of example and without limitation, magnesium stearate, calcium stearate, talc, glyceryl behenate, poly(ethylene glycol), hydrogenated vegetable oil, mineral oil, stearic acid, combinations thereof and other such materials known to those of ordinary skill in the art.

As used herein, the term “binder” is intended to mean substances used to cause adhesion of powder particles in solid formulations. Such compounds include, by way of example and without limitation, acacia, alginic acid, tragacanth, carboxymethylcellulose sodium, poly(vinylpyrrolidone), compressible sugar (e.g., NuTab), ethylcellulose, gelatin, liquid glucose, methylcellulose, povidone and pregelatinized starch, combinations thereof and other materials known to those of ordinary skill in the art. When needed, other binders may also be included in the present formulation. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “diluent” or “filler” is intended to mean inert substances used as fillers to create the desired bulk, flow properties, and compression characteristics in the preparation of solid dosage forms. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “direct compression excipient” is intended to mean a compound used in direct compression formulations. Such compounds include, by way of example and without limitation, dibasic calcium phosphate (e.g. Ditab™), microcrystalline cellulose, direct compression lactose (e.g. Tablettose™, Lactose DT), combinations thereof and other such materials known to those of ordinary skill in the art.

As used herein, the term “glidant” is intended to mean agents used in tablet and capsule formulations to improve flow-properties during compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the term “lubricant” is intended to mean substances used in solid formulations to reduce friction during compression. Such compounds include, by way of example and without limitation, calcium stearate, magnesium stearate, mineral oil, stearic acid, zinc stearate, combinations thereof and other such materials known to those of ordinary skill in the art.

As used herein, the term “opaquant” is intended to mean a compound used in tablet coatings or capsules providing useful opacity which can aid the stability to the light in case of sensitive agents. It may be used alone or in combination with a colorant. Such compounds include, by way of example and without limitation, titanium dioxide and other such materials known to those of ordinary skill in the art.

As used herein, the term “polishing agent” is intended to mean a compound used to impart brightness to the surface of particles or dosage forms. Such compounds include, by way of example and without limitation, carnauba wax, white wax, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, croscarmellose sodium, sodium starch glycolate, crospovidone, starches such as corn starch, potato starch, pre-gelatinized and modified starches thereof, sweeteners, clays, such as bentonite, microcrystalline cellulose (e.g. Avicel™), carboxymethylcellulose calcium, cellulose polyacrylin potassium (e.g. Amberlite™), alginates, sodium starch glycolate, gums such as agar, guar, locust bean, karaya, pectin, tragacanth, combinations thereof and other such materials known to those of ordinary skill in the art.

As used herein, the term “colorant” is intended to mean a compound used to impart color to pharmaceutical preparations. Such compounds include, by way of example and without limitation, FD&C Red No. 3, FD&C Red No. 20, FD&C Yellow No. 6, FD&C Blue No. 2, FD&C Green No. 5, FD&C Orange No. 5, FD&C Red No. 8, caramel, and iron oxide (black, red, yellow), other FD&C dyes and natural coloring agents such as grape skin extract, beet red powder, beta-carotene, annato, carmine, turmeric, paprika, combinations thereof and other such materials known to those of ordinary skill in the art.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors, which have been found to be particularly useful, include commercially available orange, grape, cherry and bubble gum flavors and mixtures thereof. The amount of flavoring may depend on a number of factors, including the desired organoleptic effect. Flavors will be present in any amount as desired by the artisan of ordinary skill in the art. Particularly preferred flavors are the grape and cherry flavors and citrus flavors such as orange.

The delivery device of the invention can also include oils such as fixed oils, peanut oil, sesame oil, cottonseed oil, corn oil and olive oil; fatty acids such as oleic acid, stearic acid and isostearic acid; and fatty acid esters such as ethyl oleate, isopropyl myristate, fatty acid glycerides and acetylated fatty acid glycerides. The device can also include alcohol such as ethanol, isopropanol, hexadecyl alcohol, glycerol and propylene glycol; glycerol ketals such as 2,2-dimethyl-1,3-dioxolane-4-methanol; ethers such as poly(ethylene glycol) 450; petroleum hydrocarbons such as mineral oil and petrolatum; water; a pharmaceutically suitable surfactant, suspending agent or emulsifying agent; or mixtures thereof.

Soaps and synthetic detergents may be employed as surfactants and as vehicles for detergent compositions. Suitable soaps include fatty acid alkali metal, ammonium, and triethanolamine salts. Suitable detergents include cationic detergents such as dimethyl dialkyl ammonium halides, alkyl pyridinium halides, and alkylamine acetates; anionic detergents such as alkyl, aryl and olefin sulfonates, alkyl, olefin, ether and monoglyceride sulfates, and sulfosuccinates; non-ionic detergents such as fatty amine oxides, fatty acid alkanolamides, and poly(oxyethylene)-block-poly(oxypropylene) copolymers; amphoteric detergents such as alkyl β-aminopropionates and 2-alkylimidazoline quaternary ammonium salts; and mixtures thereof.

Various other components, not otherwise listed above, can be added to the present formulation to provide a device with a desired release profile. Such components include, by way of example and without limitation, glycerolmonostearate, nylon, cellulose acetate butyrate, d,l-poly(lactic acid), 1,6-hexanediamine, diethylenetriamine, starches, derivatized starches, acetylated monoglycerides, gelatin coacervates, poly(styrene-maleic acid) copolymer, glycowax, castor wax, stearyl alcohol, glycerol palmitostearate, polyethylene, poly(vinyl acetate), poly(vinyl chloride), 1,3-butylene-glycoldimethacrylate, ethyleneglycol-dimethacrylate and methacrylate hydrogels.

It should be understood that the compounds used in the art of pharmaceutical formulation generally serve a variety of functions or purposes. Thus, if a compound named herein is mentioned only once or is used to define more than one term herein, its purpose or function should not be construed as being limited solely to that named purpose(s) or function(s).

As used herein, the term “tramadol” is taken to mean all known forms of tramadol unless otherwise specified. Tramadol can be present in racemic, optically pure or optically enriched forms. The tramadol can also be present as the pharmaceutically acceptable salt form or free-base forms. As used herein, “pharmaceutically acceptable salt” refers to tramadol that has been modified by reacting it with an acidifying agent as needed to form an ionically bound pair. Examples of pharmaceutically acceptable salts include conventional non-toxic salts formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art or described herein. The salts can be prepared from acidifying agents that are organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. Lists of other suitable salts are found in Remington's Pharmaceutical Sciences, 17^{th}. ed., Mack Publishing Company, Easton, Pa., 1985, p. 1418, the relevant disclosure of which is hereby incorporated by reference. Preferred salt forms of tramadol include the hydrochloride (Degussa Chemical), acetate, succinate, citrate, tartrate, malate, phosphate, pyrophosphate, sulfate, maleate and fumarate salts of tramadol.

The tramadol can be included in the compositions and dosage form of the invention as either a free base or a salt. When tramadol is included in the free base, an acid acidifying agent can be included in the composition or dosage form. After exposure of the composition to an aqueous environment, the acid would dissolve and contact the tramadol in situ thereby forming a salt of the tramadol and aiding in its dissolution such that the tramadol salt would be released from the composition.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The amount of tramadol incorporated in each unit dose of the invention will be at least one or more dosage forms and can be selected according to known principles of pharmacy. An effective amount of therapeutic compound is specifically contemplated. By the term “effective amount”, it is understood that, with respect to, for example, pharmaceuticals, a pharmaceutically effective amount is contemplated. A pharmaceutically effective amount is the amount or quantity of tramadol which is enough for the required or desired therapeutic response, or in other words, the amount, which is sufficient to elicit an appreciable biological response when, administered to a patient. The appreciable biological response may occur as a result of administration of single or multiple unit doses of an active substance. A unit dose may comprise one or more dosage forms, such as capsules. It will be understood that the specific dose level for any patient will depend upon a variety of factors including the indication being treated, severity of the indication, patient health, age, sex, weight, diet, pharmacological response, the specific dosage form employed and other such factors.

The recommended daily dose of tramadol HCl is from 50 and 100 mg every 4 to 6 hours, with a maximum dose of 400 mg/day. The multi-particulate composition and dosage form of the invention comprises a first amount of tramadol in rapid release particulate form and a second amount of tramadol in controlled release particulate form. Based upon the total combined mass of rapid and controlled release particles in a unit dose being 100%, the first amount of rapid release particles comprises from 0 to 100 mg, >0 to 100 mg, or 25 to 75 mg of tramadol HCl, and the second amount of controlled release particles comprises from 50 to 400 mg of tramadol HCl or 75-225 mg of tramadol HCl. Typically, the first amount is 20-30% by wt. of the total mass of particles (total rapid release and controlled release particles) in the unit dose, and the second amount is 80-70%, respectively, of the total amount of particles in the unit dose.

The amount of tramadol present in the individual particles can vary from 20% to 90% by wt. of the particle. In some embodiments, the amount of tramadol ranges from 30% to 60% by wt., 20% to 60%, or from 40% to 50% by wt. of the weight of the particle. The spheronization method used can easily incorporate drug concentrations lower than 50% by wt.; however, the amounts of particles needed for normal dosing become larger. The amount of tramadol present in the particle may depend upon the spheronization technique used to prepare the particles. Rotogranulation may be used to make particles comprising up to about 90% by wt. of tramadol or tramadol salt.

The particulate composition comprised within the rapid particles and the uncoated core of the controlled release particles can be prepared according to the methods disclosed herein or by any comparable suitable means known for the preparation of pharmaceutical particulates. Exemplary methods include dry granulation, wet granulation, extrusion, spheronization, or combinations thereof. In an exemplary extrusion/spheronization process, tramadol and excipients are mixed with a liquid to form a uniform or homogeneous mixture having a doughy consistency. Water is added slowly with mixing to the point where the material begins to produce balls during mixing. The doughy mass is then extruded using an extruder equipped with a screen having an array of about 1 mm holes to form stranded extrudates. The strands are then broken into particles having a length approximating the diameter of the strands. In a marumerizer, the particles are rounded (spheronized). The particles are then dried using, for example, a tray dryer, tumble dryer, cone dryer, microwave dryer, or a fluid bed dryer.

It may be useful to include a spheronization aid when employing a spheronization process. A spheronization aid is a material or combination of materials included within the matrix of a particle to aid in formation of spheres during the spheronization of the particles. Suitable materials include cellulose, such as microcrystalline cellulose (MC), suitable grades of which can be purchased under the trademark AVICEL™ (FMC Biopolymer,). Particular grades of AVICEL include the PH-101, PH-105, RC-581, RC-591, and CL-611 grades. The PH grades of AVICEL™ comprise microcrystalline cellulose. The pH grades differ as follows.

AVICEL Grade

| | PH-101 | PH-105 |
|---|---|---|
| Moisture (%) | 3.0-5.0 | NMT 5.0 |
| Nominal Particle Size | 50 | 20 |
| (μm) | | |
| Loose Bulk Density (g/cc) | 0.26-0.31 | 0.20-0.20 |

NMT denotes “not more than”.

The RC and CL grades of AVICEL™ comprise an attritted mixture of microcrystalline cellulose and sodium carboxymethylcellulose (CMC). AVICEL™RC-581, RC-591 and CL-611 are listed as microcrystalline cellulose and carboxymethylcellulose sodium, in the U.S. Pharmacopeia/National Formulary and as dispersible cellulose in the British Pharmacopoeia. The RC and CL grades differ as follows.

| | AVICEL Grade | | |
|---|---|---|---|
| | RC-581 | RC-591 | CL-611 |
| Loss on Drying (%) | NMT 6.0 | NMT 6.0 | NMT 6.0 |
| Heavy Metals (%) | NMT 0.001 | NMT 0.001 | NMT 0.001 |
| NaCMC (%) | 8.3-13.8 | 8.3-13.8 | 11.3-18.8 |
| Sieve Fraction (wt %) | | | |
| 60 mesh: | NMT 0.1 | NMT 0.1 | NMT 0.1 |
| +200 mesh: | NMT 35 | | |
| +325 mesh: | | NMT 45 | NMT 50 |
| Ph | 6.0-8.0 | 6.0-8.0 | 6.0-8.0 |
| Viscosity, (cps) | 72-168 | 39-91 | 50-118 |
| | @ 1.2% solids | @ 1.2% solids | @ 2.6% solids |
| Residue on ignition | NMT 5.0 | NMT 5.0 | NMT 5.0 |
| (%) | | | |

NMT denotes “not more than”.

A spheronization aid can be present in an amount of about 20% to 80% by wt, or about 30-50% by wt. based upon the weight of the composition within which it is present.

A binder, as described herein, can be included in the particles. A particularly suitable binder is starch. One specific grade of starch is Starch 1500 (Colorcon); however, other grades, forms and types of starch can also be used. Suitable starch can be obtained from suppliers such as Ultrachem Ltd. (UK), Cerestar (Mechelen, Belgium, Europe), National Starch & Chemical (Bridgewater, N.J.), American Maize Products (Hammond, Ind.).

If desired, the particles or dosage form of the invention can be coated with a finish coating as is commonly done in the art to provide the desired shine, color, taste or other aesthetic characteristics. Materials suitable for preparing the finish coating are well known to those of ordinary skill in the art.

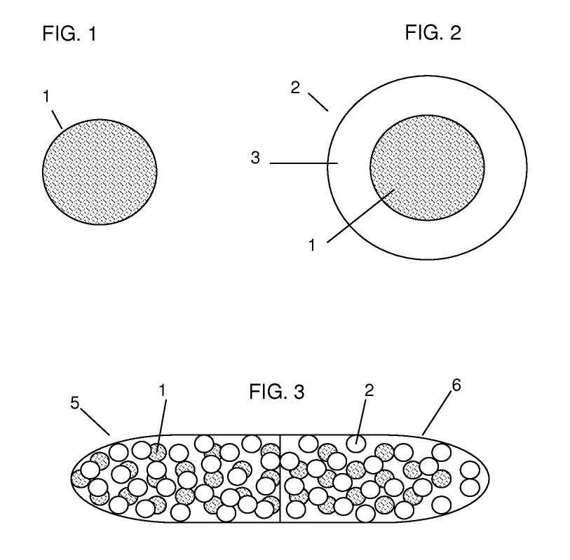

FIG. 1 depicts a cross-sectional view of an exemplary rapid release particle (1) made according to the invention. The particle comprises tramadol dispersed within a matrix of excipient(s). The particle is depicted as being spherical; however, any particle shape can be used. Accordingly, the particles of the invention can be shaped as rods, beads, pellets, spheres, ellipsoids, dumbbells or other known particle shapes used in the pharmaceutical industry. In some embodiments, the controlled release particles are spherically shaped because that shape requires the least amount of coating per unit surface area.

The diameter or length of the coated or uncoated particles generally ranges from 0.5 to 3 mm or from about 1 to about 1.4 mm. These size ranges are preferred so as to provide a balance between content uniformity, when the beads are included in a dosage form or pharmaceutical composition, and particle size. Generally, the smaller the particle size, the easier it is to obtain a more reproducible content uniformity; however, smaller particles generally require more total coating weight per batch of particles to achieve the same tramadol release profile as larger particles. This is because the smaller the particle size, the thicker the coating required to give the same release profile as a bigger particle.

FIG. 2 depicts a cross-sectional view of an exemplary controlled release particle (2) made according to the invention. In this embodiment, the controlled release particles (2) are prepared by coating (3) a portion of rapid release particles (1) with a film-forming composition as described herein. The film-forming composition comprises a semipermeable polymer (a film-forming polymer or combination of polymers that forms a semipermeable membrane when applied to a core and dried), pore former and plasticizer. The weight, and thereby the thickness, of the coating with respect to the weight of the otherwise uncoated core (the rapid release particle) can be varied as detailed herein to provide controlled release particles having a desired drug release profile. Generally, the weight of the coating ranges from 5 to 50% by wt. or 10 to 25% by wt. based upon the weight of the coated particle.

FIG. 3 depicts a side elevation of a capsule dosage form (5) comprising a capsule shell (6) encasing a population of rapid release particles (1) and a population of controlled release particles (2). The uncoated rapid release particles typically have a smaller diameter than the coated controlled release particles when the rapid release particles are used as the core of the controlled release particles; however, such a relationship in particle size is not required in order to practice the invention. A coated particle on average will have a slightly larger diameter than the corresponding uncoated particle from which it was made due to the presence of the surface coating.

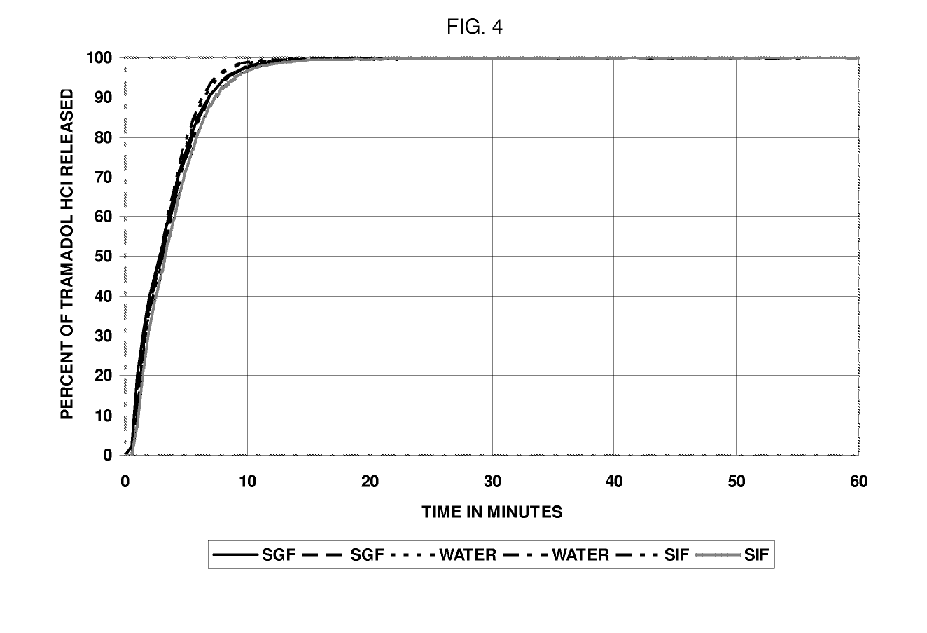

The in vitro release profile of rapid release particles prepared according to Example 1 was evaluated according to the method of Example 4. FIG. 4 depicts the release profile for exemplary rapid release particles determined in water, SIF or SGF. The results indicate that release is completed within 20 min or less 15 min following exposure of the particles to the aqueous medium. The results also indicate that release of tramadol from these particles is substantially pH independent. The USP defines rapid release dosage forms in terms of the time for 90% of the dose to be released. In general if a dosage form releases 90% of the drug in 45 minutes, it falls under the definition of a rapid release dosage formulation.

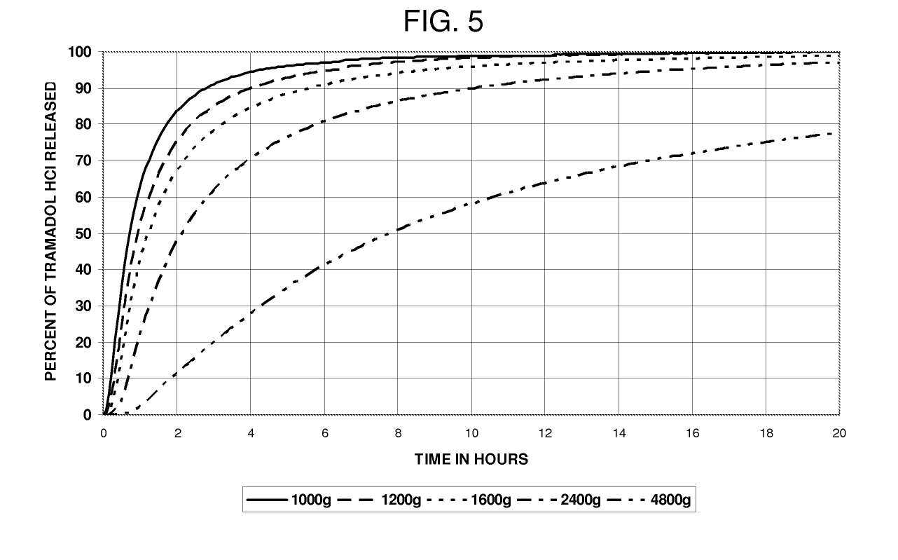

The in vitro release profile of controlled release particles prepared according to Example 2 was evaluated according to the method of Example 4. FIGS. 5, 6 and 8 depict the release profile for exemplary controlled release particles determined in water. The drug release profiles depicted in FIG. 5 demonstrate the effect of different amounts of coating (see legend) applied to a batch of tramadol HCl particles at 40% drug loading. The greater the amount of coating applied to the particles, the thicker the coating and the slower the drug release.

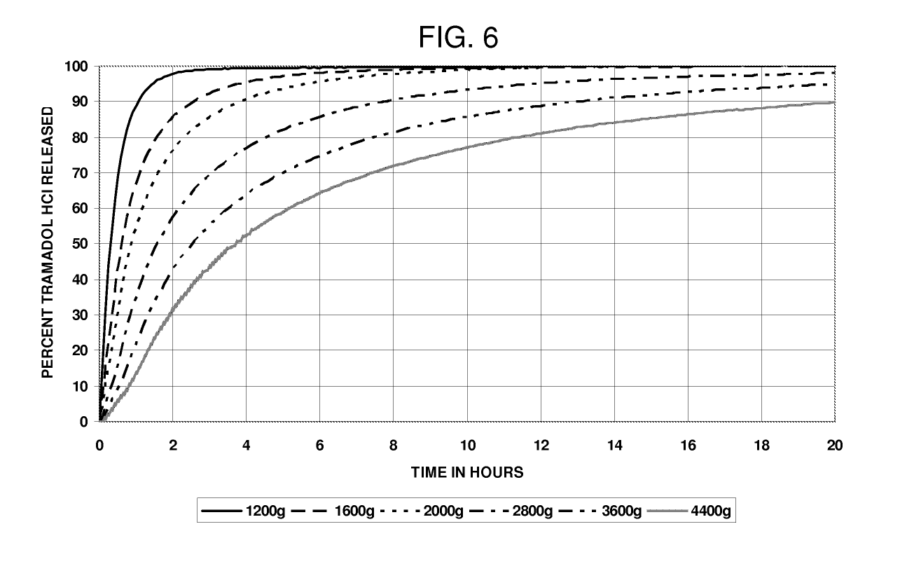

FIG. 6 depicts the change in the tramadol release profile that might be expected by increasing the thickness of the coating on the controlled release particles. A thicker coating provides a reduced rate of release of tramadol and a slight delay in the initial release of tramadol as compared to an otherwise similarly formulated coating. Accordingly, the thickness of the coating is sufficient to reduce the rate of tramadol release enough to provide a continuous extended release over a period of not less than 10, not less than 12, not less than 14, not less than 18 or not less than 20 hours following exposure to an aqueous environment or following administration. However, the thickness of the coating will be thin enough that completion of tramadol release from the controlled release particles will occur not more than 36 hours, not more than 32, hours, not more than 28 hours, or not more than 24 hours following exposure to an aqueous environment or following administration. The particles of FIG. 6 comprise a 50% by wt. drug loading in contrast to those of FIG. 5 which comprise a 40% by wt. drug loading. By varying the amount of coating on a particle, the drug release can extend over from 10 to >24 hours. In some embodiments, the coating is thin enough such that greater than 90% of the drug is released in less than 24 hours when the coated particle is placed in an aqueous environment. In some embodiments, the thickness of the coating is about 20 to 300 μm, or about 50 to 150 μm thick.

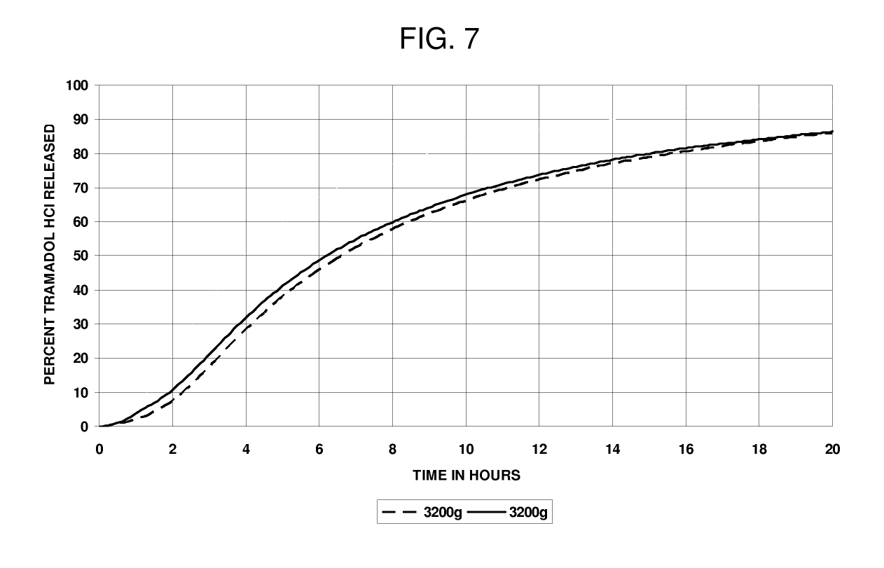

FIG. 7 depicts the release profile for exemplary controlled release particles determined in water. The results indicate that release is completed following exposure of the particles to the aqueous medium. The data demonstrate that sufficient coating can be applied to extend the release of tramadol HCl to periods greater than 20 hours.

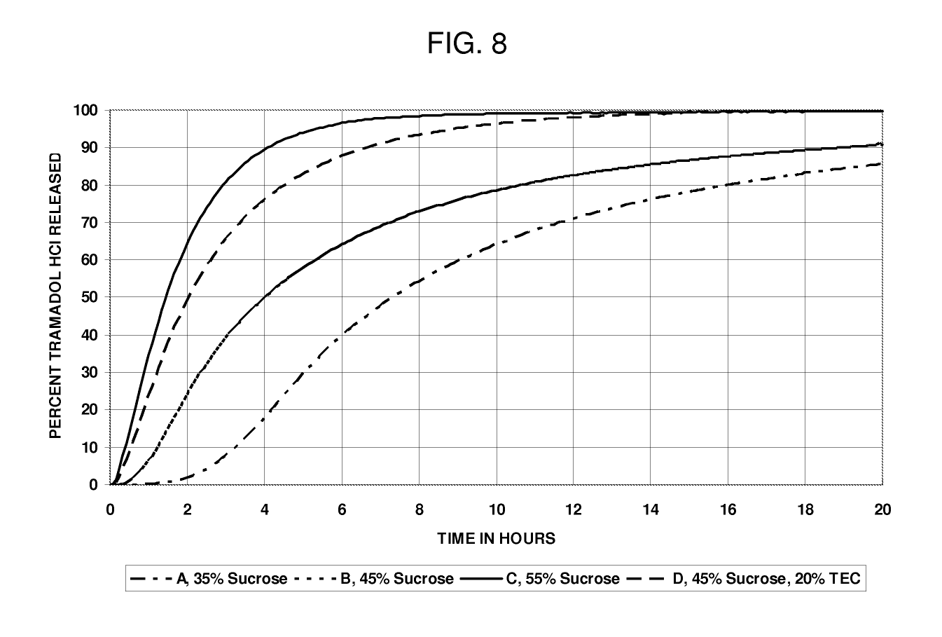

The data depicted in FIG. 8 demonstrates the effect that the additional pore former (sucrose) has upon the rate of tramadol HCl release from the controlled release particle. The dash-dot line (A) represents data for 35% sucrose (expressed as percentage of CAB 171-15PG), the short dotted line (B) represents data for 45% sucrose, and the solid line (C) represents data for 55% sucrose. These three coatings contained 30% triethyl citrate (as % of CAB 171-15PG). The long dashed line (D) represents data demonstrating the effect of Triethyl Citrate on the release of tramadol HCl. The TEC is reduced to 20% with sucrose at 45%. The rate of release increases with decreasing amount of plasticizer. This indicates that TEC is a good plasticizer for CAB 171-15PG. Without being held to a particular mechanism, it is believed that the reason given the rate is faster with lower amounts of plasticizer is that with less TEC the polymer is not able to surround and block off the ability of the sucrose to exit the coating as well when the polymer is more highly plasticized at 30% TEC. The result is greater overall porosity in the coating.

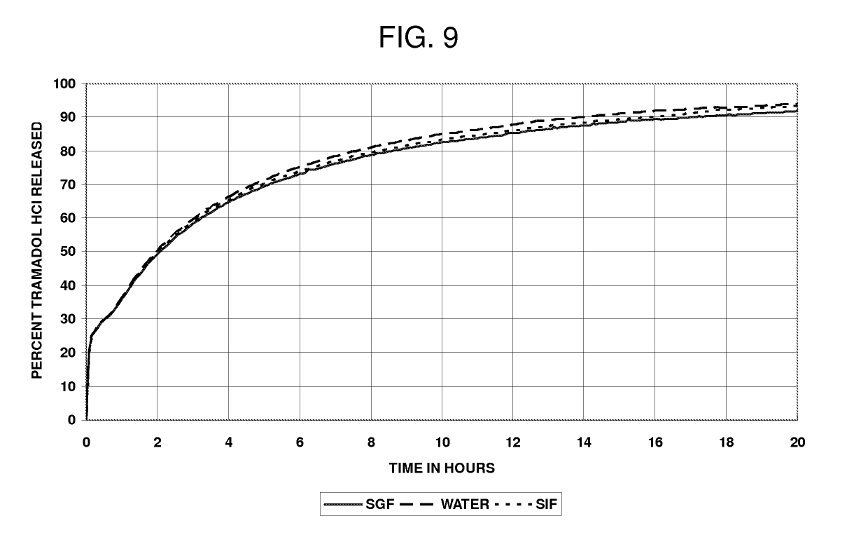

When the rapid release particles and controlled release particles are combined to form an extended release pharmaceutical composition or dosage form in a capsule, the tramadol will be released substantially continuously over an extended period of time beginning after wetting of the particles, which occurs after a sufficient amount of the shell has dissolved to permit exposure of the particles to an aqueous environment. In general, release of drug will begin within about 30 min after administration (or exposure to an aqueous environment) and end at about 10 to 24 hours after administration (or exposure to an aqueous environment). FIG. 9 depicts the combination in vitro release profile provided by an extended release capsule dosage form prepared according to Example 3, Method B. Within 15 min after exposure to an aqueous fluid, the dosage form has released about 25% of the tramadol charge present therein, and over the following 18 to 24 hours, the dosage form completes its release of tramadol charge (the remaining 75%). The results indicate that release of tramadol is substantially pH independent. In some embodiments, the drug release profile for controlled release particles are approximately first order.

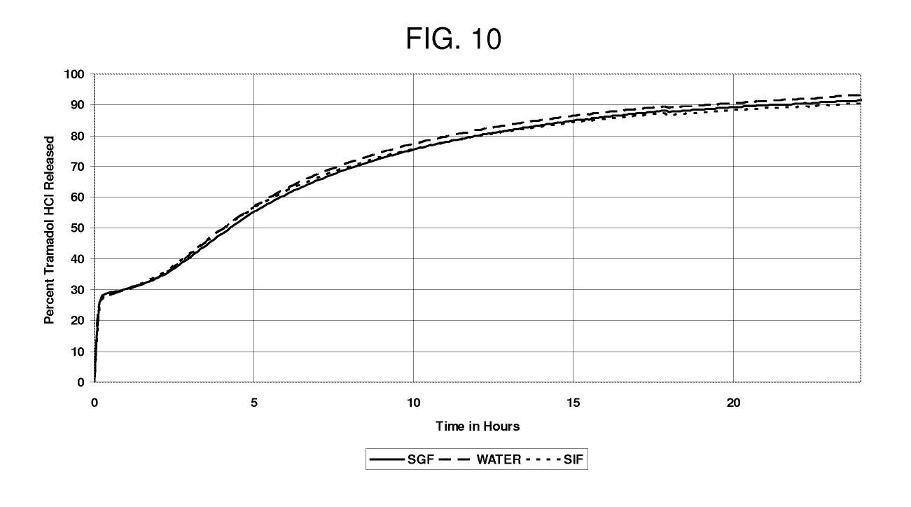

FIG. 10 depicts the combination in vitro release profile provided by an extended release capsule dosage form prepared according to Example 3, Method C. Within 15 min after exposure to an aqueous fluid, the dosage form has released about 25% to 30% of the tramadol charge present therein, and over the following 20 to 30 hours, the dosage form completes its release of tramadol charge (the remaining 75% to 70%, respectively). The results indicate that release of tramadol is substantially pH independent.

The invention provides an extended release dosage form comprising: a population of rapid release particles comprising a first charge of tramadol and at least one excipient; a population of controlled release particles comprising a core and a semipermeable coating enclosing the core, the core comprising a second charge of tramadol and at least one excipient, and the coating comprising a film-forming semipermeable polymer, a pore former and a plasticizer; wherein the dosage form releases tramadol over (throughout) an extended period of time in a substantially pH independent manner.

The amount of rapid release particles and controlled release particles to be included in the extended release dosage form will depend upon the desired pharmacokinetic performance or clinical effect to be provided by the dosage form. If a higher initial loading dose (plasma concentration) of tramadol is desired, then the dosage form will contain a higher relative percentage of rapid release particles. If a lower initial loading dose (plasma concentration) of tramadol is desired, then the dosage form will contain a lower relative percentage of rapid release particles. Likewise, if a higher maintenance plasma concentration of tramadol is desired, then the dosage form will contain a higher relative percentage of controlled release particles. If a lower maintenance plasma concentration of tramadol is desired, then the dosage form will contain a lower relative percentage of controlled release particles.

The actual amount of rapid release particles and controlled release particles to be included in a target extended release dosage can be determined by calculating the concentration of tramadol present in each population of particles and adding in the required amount of each type of particle to provide the desired target extended release dosage form. For example, a target capsule might contain 50 mg of tramadol HCl in rapid release form and 150 mg of tramadol HCl in controlled release form. In this case, the weight of rapid release particles that is equivalent to (or contains) 50 mg of tramadol HCl is determined, and the weight of controlled release particles that is equivalent to (or contains) 150 mg of tramadol HCl is determined. The amounts of rapid release particles and controlled release particles are then combined and placed in the capsule.

One can determine the amount of particles (rapid release or controlled release) that is equivalent to a target weight of tramadol by following the method detailed in Example 6. In this case, the drug loading (expressed as % by wt., or as mg of tramadol per 100 mg of particles) for a population of particles is determined. For example, if a batch of rapid release particles has a drug loading of 50% (50 mg of tramadol HCl per 100 mg of particles), then 100 mg of particles would need to be added to the capsule in order to provide 50 mg of tramadol HCl.

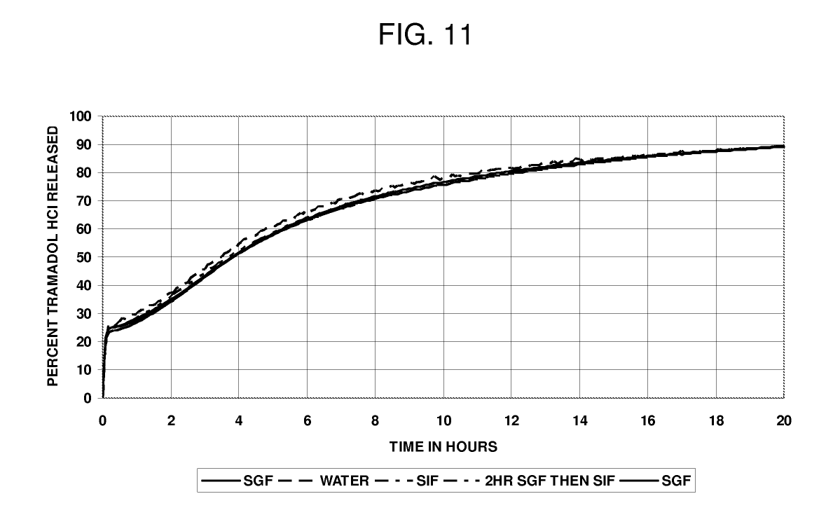

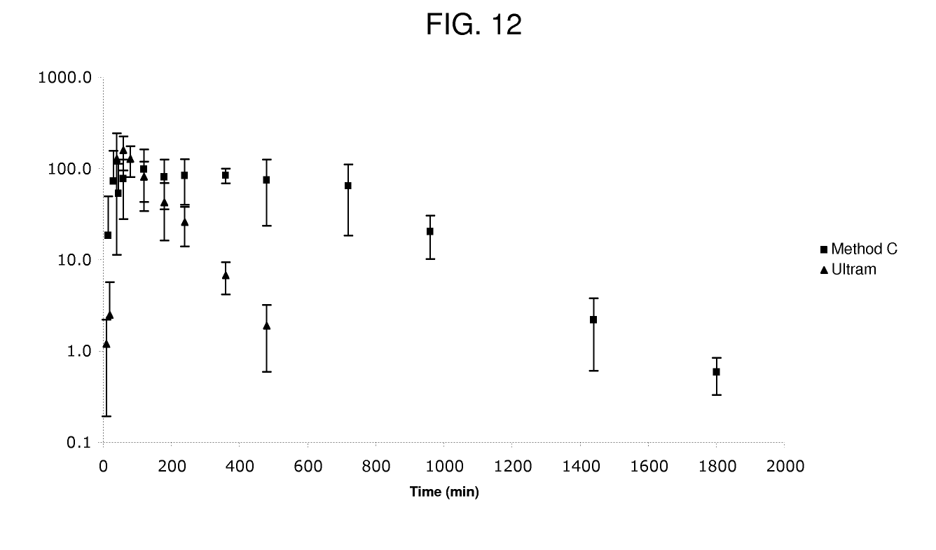

FIG. 11 depicts the combination in vitro release profile provided by an extended release capsule dosage form prepared according to Example 3, Method D. Within 15 min after exposure to an aqueous fluid, the dosage form has released about 25% of the tramadol charge present therein, and over the following 20 to 30 hours, the dosage form completes its release of tramadol charge (the remaining 75%). The results indicate that release of tramadol is substantially pH independent. In the process used to prepare the controlled release portion of the dosage form, no filler beads were employed.

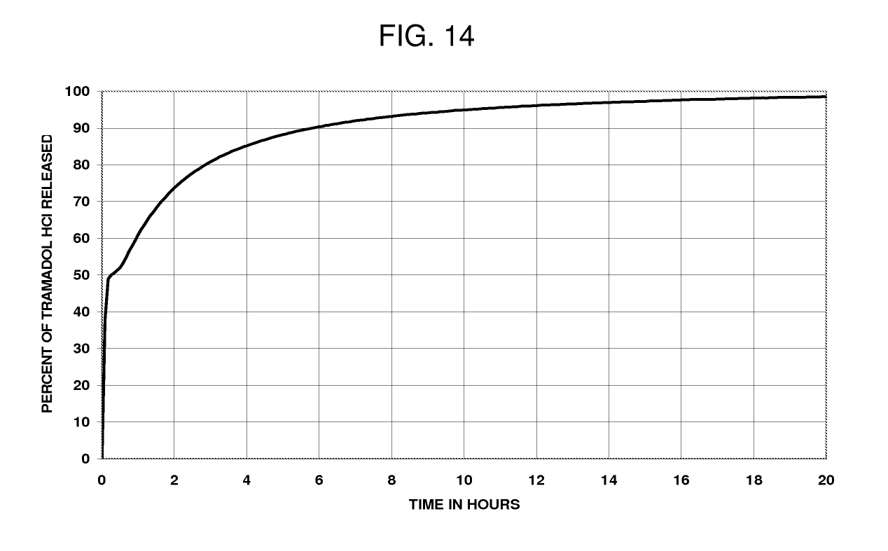

FIG. 14 depicts the combination in vitro release profile provided by an extended release capsule dosage form containing 100 mg of tramadol HCl in immediate release form (using the particles of FIG. 4) and 100 mg of tramadol HCl in controlled release (using the particles of FIG. 5 denoted as the 2400 g coating lot). The profile indicates a rapid increase in the amount of tramadol present in solution followed by a more gradual increase until completion of release of tramadol. This particular batch of controlled release particles are particularly suitable for twice-daily dosing due to the almost complete release (>95%) of tramadol within twelve hours after exposure to an aqueous environment.

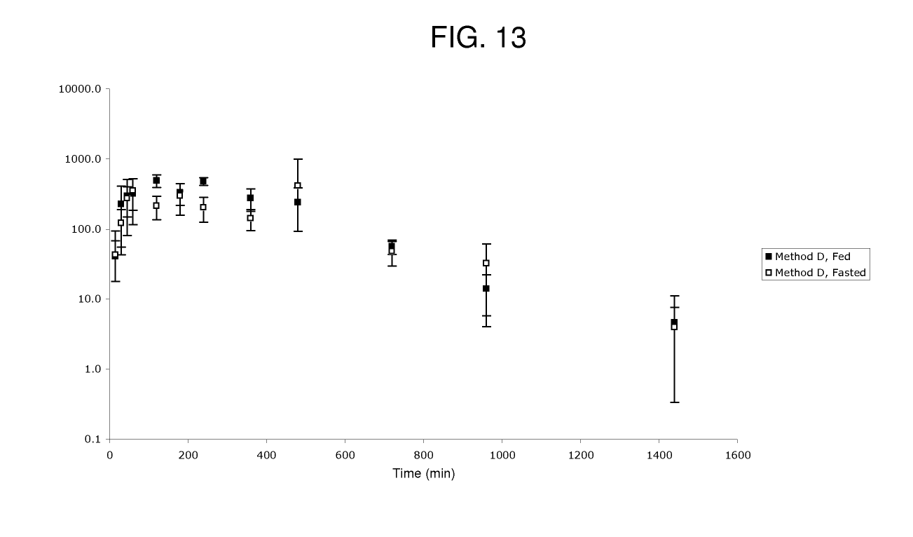

The in vivo performance of the extended release dosage form was evaluated according to Example 5, Trials A and B. Extended release capsules of the invention were administered orally to beagles and the concentration of tramadol present in their plasma determined periodically following administration. The plasma concentration profile for tramadol from Example 5, Trial A is depicted in FIG. 12. The data demonstrates that the rapid release portion of the present invention provides early plasma levels equivalent to that of a unit dose of ULTRAM and that controlled release particle provide elevated or sustained plasma levels of tramadol for a period of more than 12 hours. The plasma concentration profile for tramadol from Example 5, Trial B is depicted in FIG. 13. The data demonstrates that the present invention exhibits sustained plasma levels of tramadol in both the fed and fasted states.

If a dosage form comprising 50 mg of tramadol (free base or salt) in rapid release particles and 150 mg of tramadol (free base or salt) in controlled release particles is administered to a human subject, one might expect plasma tramadol levels to rise rapidly through the first sixty minutes post administration to levels between 100 and 450 ng of tramadol/ml of plasma. Subsequently, mean plasma tramadol levels might remain above 100 ng/ml for at least 12 hours post administration. That said, a dosage form of the invention comprising rapid and controlled release particles of tramadol as described herein can be adapted to provide a wide range of different plasma profiles for tramadol.

In view of the above description and the examples below, one of ordinary skill in the art will be able to practice the invention as claimed without undue experimentation. The foregoing will be better understood with reference to the following examples that detail certain procedures for the preparation of embodiments of the present invention. All references made to these examples are for the purposes of illustration. The following examples should not be considered exhaustive, but merely illustrative of only a few of the many embodiments contemplated by the present invention.

### Example 1

### Preparation of Rapid Release Particles

### Method A. Preparation of Tramadol HCl Beads at 40% Drug Loading.

The following ingredients were provided in the amounts indicated.

| Ingredient | Amount (g) |
|---|---|
| Tramadol HCl | 80 |
| Avicel PH-101 | 96 |
| Starch 1500 | 24 |

The solids were mixed and granulated with 44 g of water in a planetary mixer over 20 minutes with slow addition of the water. The wet dough was extruded using a Nica E-140 extruder with a 1.2 mm screen. The extrudate was added to a Luwa QJ-230 marumerizer with a 2 mm V-grooved plate rotating at 1000 rpm and spheronized for 5 minutes. The spheres were tray dried at RT. The yield was 72% for beads in the range of 1 to 1.4 mm. The in vitro release profile for tramadol was determined according to Example 4. The drug release was pH independent and >95% drug was released within 10 minutes after exposure to the aqueous assay solution.

### Method B. Preparation of Tramadol HCl Beads at 50% Drug Loading.

The following ingredients were provided in the amounts indicated.

| Ingredient | Amount (g) |
|---|---|
| Tramadol HCl | 150 |
| Avicel PH-101 | 120 |
| Starch 1500 | 30 |

The solids were mixed and granulated with 67 g of water in a planetary mixer over 20 minutes with slow addition of the water. The wet dough was extruded using a Nica E-140 extruder with a 1.2 mm screen. The extrudate was added to a Luwa QJ-230 marumerizer with a 2 mm V-grooved plate rotating at 1000 rpm and spheronized for 10 minutes. The spheres were tray dried at RT. The yield was 37% for beads in the range of 1 to 1.4 mm. The in vitro release profile for tramadol was determined according to Example 4. The drug release was pH independent and >95% drug was released by 10 minutes.

### Method C. Preparation of Tramadol HCl Beads at 60% Drug Loading.

The following ingredients were provided in the amounts indicated.

| Ingredient | Amount (g) |
|---|---|
| Tramadol HCl | 180 |
| Avicel PH-101 | 96 |
| Starch 1500 | 24 |

The solids were mixed and granulated with 56 g of water in a planetary mixer over 20 minutes with slow addition of the water. The wet dough was extruded using a Nica E-140 extruder with a 1.2 mm screen. The extrudate was added to a Luwa QJ-230 marumerizer with a 2 mm V-grooved plate rotating at 1000 rpm. The material formed only very large beads. The material was collected and extruded again and the extrudate let dry for 5 minutes. The extrudate was added to the marumerizer where it broke up but would not spheronize. Some water from an atomizing bottle was sprayed on the material, which was then spheronized for 15 minutes. The spheres were tray dried at RT. The yield was 57% for beads in the range of 1 to 1.4 mm.

### Method D. Preparation of Tramadol HCl Beads at 50% Drug Loading Using AVICEL™ RC-581.

The following ingredients were provided in the amounts indicated.

| Ingredient | Amount (g) |
|---|---|
| Tramadol HCl | 150 |
| Avicel RC-581 | 120 |
| Starch 1500 | 30 |

The solids were mixed and granulated with 82 g of water in a planetary mixer over 20 minutes with slow addition of the water. The wet dough was extruded using a Nica E-140 extruder with a 1.0 mm screen. The extrudate was added to a Luwa QJ-230 marumerizer with a 2 mm V-grooved plate rotating at 1000 rpm. The material formed only very large beads. The material was collected and extruded again and the extrudate let dry for 5 minutes. The extrudate was added to the marumerizer where it broke up but would not spheronize. Water from an atomizing bottle was sprayed on the material, and it was spheronized for 15 minutes. The beads were not very spherical but dumbbell shaped. The spheres were tray dried at RT. The yield was 71% for beads in the range of 1 to 1.4 mm.

### Method E. Preparation of Tramadol HCl Beads at 50% Drug Loading Using AVICEL™ RC-581 and AVICEL™ PH-101.

The following ingredients were provided in the amounts indicated.

| Ingredient | Amount (g) |
|---|---|
| Tramadol HCl | 150 |
| Avicel RC-581 | 90 |
| Avicel PH-101 | 60 |

The solids were mixed and granulated with 74 g of water in a planetary mixer over 20 minutes with slow addition of the water. The wet dough was extruded using a Nica E-140 extruder with a 1.2 mm screen. The extrudate was added to a Luwa QJ-230 marumerizer with a 2 mm V-grooved plate rotating at 1000 rpm. The material formed only very large beads. The material was collected and extruded again. The extrudate was added to the marumerizer where it broke up and spheronized. The bulk of the beads were somewhat larger than 1.4 mm. The spheres were tray dried at RT. The yield was 34% for beads in the range of 1 to 1.4 mm.

### Method F. Preparation of Tramadol HCl Beads at 50% Drug Loading Using AVICEL™ RC-581 and AVICEL PH-101.

The following ingredients were provided in the amounts indicated.

| Ingredient | Amount (g) |
|---|---|
| Tramadol HCl | 150 |
| Avicel RC-581 | 75 |
| Avicel PH-101 | 75 |

The solids were mixed and granulated with 64 g of water in a planetary mixer over 20 minutes with slow addition of the water. The wet dough was extruded using a Nica E-140 extruder with a 1.0 mm screen. The extrudate was added to a Luwa QJ-230 marumerizer with a 2 mm V-grooved plate rotating at 1000 rpm. The extrudate was added to the marumerizer where it broke up and spheronize. The bulk of the beads were between 1 and 1.4 mm. The spheres were tray dried at RT. The yield was 72% for beads in the range of 1 to 1.4 mm.

The rapid release particles prepared according to this example are used in combination with the controlled release particles of Example 2 to form a multi-particulate composition of the invention.

### Example 2

### Preparation of Controlled Release Particles

### Method A. Coating of Beads Prepared According to Example 1.

The following ingredients were provided in the amounts indicated.

| Ingredient | Amount | |
|---|---|---|
| Cellulose Acetate Butyrate CAB 381-20 | 72 | g |
| (semipermeable polymer) | | |
| Sucrose (pore former) | 25.2 | g |
| Triethyl citrate (plasticizer) | 21.6 | g |
| Acetone (coating solvent) | 2.0 | L |
| Ethanol (coating solvent) | 400 | ml |
| Water (coating solvent) | 400 | ml |

Several batches of the coating solution were prepared and 40 g of beads (1.2 to 1.4 mm) were coated in a UniGlatt fluid bed coater using 800 g of filler beads (0.7 to 0.84 mm). Filler beads were used because the UniGlatt coater employed requires about 800 to 900 g of beads for a coating run. This would require nearly 500 g of Tramadol HCl for a single coating run. To conserve on drug and time to prepare the beads it is convenient and cost effective to use filler beads to make up the coating charge. The active beads can be separated from the filler beads by sieving using standard mesh screens.

The operating conditions for the coater were as follows.

| Coater | UniGlatt Fluid Bed Coater |
|---|---|
| Inlet Air Temperature | 60° C. |
| Outlet Air Temperature | 38° C. |
| Atomization Air Pressure (1 mm nozzle) | 1.2 Bar |
| Coating Rate | 10 g per min |

Samples of the active beads were removed after 1200 g, 1600 g, 2000 g, 2800 g, 3600 g and 4400 g of coating solution had been applied. The release of Tramadol HCl from the beads into water (as determined in 900 mL of water at 37° C. and 50 rpm using USP dissolution apparatus No. 2) is depicted in FIG. 6. The absorption was measured in each dissolution flask every 5 minutes for 20 hours using a flow through system. The 100% value was determined by crushing the beads and measuring the amount of drug from the absorbance at 270 nm. The release rate of Tramadol HCl can be seen to decrease as the coating on the beads increases.

### Method B. Coating of Beads Prepared According to Example 1.

The following ingredients were provided in the amounts indicated.

| Ingredient | Amount | |
|---|---|---|
| Cellulose Acetate Butyrate CAB 171-15 | 216 | g |
| (semipermeable polymer) | | |
| Sucrose (pore former) | 75.6 | g |
| Triethyl citrate (plasticizer) | 64.8 | g |
| Acetone (coating solvent) | 4.0 | L |
| Ethanol (coating solvent) | 800 | ml |
| Water (coating solvent) | 800 | ml |

Fifty grams of beads (1.2 to 1.4 mm) were coated in a UniGlatt fluid bed coater using 800 g of filler beads (0.7 to 0.84 mm). The active beads can be separated from the filler beads by sieving using standard mesh screens.

The operating conditions for the coater were as follows.

| Coater | UniGlatt Fluid Bed Coater |
|---|---|
| Inlet Air Temperature | 50° C. |
| Outlet Air Temperature | 38° C. |
| Atomization Air Pressure (1 mm nozzle) | 1.5 Bar |
| Coating Rate | 10 g per min |

After 3300 g of coating solution had been applied, the active beads were separated from the filler beads. The release profile of Tramadol HCl from the beads (determined as described for Method A.) is depicted in FIG. 7.

The controlled release particles prepared according to this example are used in combination with the rapid release particles of Example 1 to form a multi-particulate composition of the invention.

### Example 3

### Preparation of Capsule Containing a Multi-Particulate Composition of Rapid Release Particles and Controlled Release Particles

### Method A. Preparing the Combined Immediate Release Beads and the Sustained Release Beads to Give the Combination Product in a Hard Gelatin Capsule.

The amount (weight 105 mg) of rapid release particles required to yield a 50 mg dose of tramadol HCl was calculated based upon the batch of rapid release particles to be used (as prepared according to Example 1). The amount (weight 401 mg) of controlled release particles required to yield a 150 mg dose of tramadol HCl was calculated based upon the batch of controlled release particles to be used (as prepared according to Example 2). The calculated amount of rapid release particles and the calculated amount of controlled release particles were combined and placed into a capsule shell and optionally sealed to provide a capsule dosage form comprising 200 mg unit dose of tramadol HCl.

### Method B. Preparing the Combined Immediate Release Beads and the Sustained Release Beads to Give the Combination Product in a Hard Gelatin Capsule.

The concentration of tramadol HCl in the uncoated beads of Example 1 (Method A) was determined to be 47.6% by wt. Therefore, it would require 105 mg of beads to yield a 50 mg dose of tramadol HCl for the immediate release portion of the combined product.

The batch of coated beads (controlled release beads) used were those of Example 2 (Method A) that received 4400 g of coating. Assay of those beads indicated a drug content of 37.4% by wt. Therefore, it would require 401 mg of these beads for a 150 mg dose of tramadol HCl.

The required amounts of the rapid release beads and controlled release beads were loaded into a No. 0 hard gelatin capsule. Several capsules were prepared for a dissolution experiment to be run in SGF, water and SIF media as described herein using water, SGF or SIF. The results are presented in FIG. 9. As can be observed, release of tramadol from the pellets is substantially pH independent.

### Method C. Preparing the Combined Immediate Release Beads and the Sustained Release Beads to Give the Combination Product in a Hard Gelatin Capsule.

The concentration of tramadol HCl in the uncoated beads Example 1 (Method B) was determined to be 49% by wt. Therefore, it would require 102 mg of beads to give a 50 mg dose of tramadol HCl for the immediate release portion of the combined product.

The coated beads used were those of Example 2 (Method B) that received 3300 g of coating. Assay of these beads indicated a drug content of 38.7% by wt. Therefore, it would require 388 mg of these beads for a 150 mg dose of tramadol HCl. The required amounts of the coated and uncoated beads were loaded into a No. 0 hard gelatin capsule. Several capsules were prepared for a dissolution experiment to be run in SGF, water and SIF media as described above. The results are presented in FIG. 10 which show an initial immediate release followed by a sustained release of tramadol HCl. The drug release shows substantially no pH dependence.

### Method D. Preparing the Combined Immediate Release Beads and the Sustained Release Beads to Give the Combination Product in a Hard Gelatin Capsule.

Several batches of particles were prepared using Example 2, Method F in order to coat a large batch without the use of filler beads. The particles used contained the particles passing 14 mesh and retained on 18 mesh (between 1.4 and 1 mm).

The concentration of tramadol HCl in the uncoated beads Example 1, Method F was determined to be 48.3% by wt. Therefore, it would require 104 mg of beads to give a 50 mg dose of tramadol HCl for the immediate release portion of the combined product.

Approximately 800 g of these uncoated beads were coated using Example 2, Method B. These beads received 2500 g of coating. Assay of these beads indicated a drug content of 39.0% by wt. Therefore, it would require 385 mg of these beads for a 150 mg dose of tramadol HCl. The required amounts of the coated and uncoated beads were loaded into No. 0 hard gelatin capsules. Several capsules were prepared for a dissolution experiment to be run in SGF, water, SIF and 2 hours in SGF then placed into SIF media for the remaining time. The results are presented in FIG. 11 that show an initial immediate release followed by a sustained release of Tramadol HCl. The drug release shows substantially no pH dependence under all conditions studied.

The particles prepared according to Example 1 and Example 2 are used in combination to form a multi-particulate composition of the invention.

### Example 4

### In Vitro Dissolution Assay

### Method A. For Rapid Release Beads.

The release of tramadol HCl from the beads was determined using USP dissolution method No. 2 with paddle rotation at 50 rpm and media at 37° C. The release was determined in water, simulated gastric fluid (no enzymes) (SGF) and in simulated intestinal fluid pH 6.8 (no enzymes) (SIF). The concentration of tramadol HCl released was determined spectrophotometrically every one-half minute for sixty minutes at a wavelength of 270 nm using a UV spectrophotometer.

### Method B. For Controlled Release Beads.

This assay was run as in Method A with the exception that the concentration of tramadol HCl was determined every 5 minutes for 20 or 24 hours.

### Example 5

### In Vivo Evaluation

Trial A. In Vivo Evaluation of Tramadol ER Dosage Form from Example 3, Method C.

Three fasted male Beagle dogs received either a two 50 mg tablets of immediate release ULTRAM™ (Ortho-McNeil Pharmaceuticals, Inc., Control 5GG103) or two 200 mg capsule dosage forms of the present invention (50 mg immediate release, 150 mg sustained release; prepared according to Example 3, Method C) followed by 30 ml of water. Blood samples were collected at predetermined time points and analyzed for tramadol and its major metabolites by a validated HPLC assay with mass detection. FIG. 12 is a plot of the average tramadol plasma concentration (±SD) as a function of time for both ULTRAM™ (-▴-) and the sustained release formulation of the present invention (-▪-). As can be seen, plasma levels for the present invention formulation rise rapidly mirroring the immediate release ULTRAM™ formulation. However, the plasma levels for the present invention formulation remain relatively constant for at least 12 hours with continued elevated tramadol levels through 24 hours.

Trial B. In Vivo Evaluation of Tramadol ER Dosage Form from Example 3, Method D.

Four male Beagle dogs received two 200 mg capsule dosage forms of the present invention (50 mg immediate release, 150 mg sustained release; prepared according to Example 3, Method D) followed by 30 ml of water. The formulations were given in either the fed or fasted state with at least a two-week wash out period between studies. In the fed studies the dogs received food immediately prior to drug administration. In all studies the dogs were fed after 8 hours and at 24 hours. Blood samples were collected at predetermined time points and analyzed for tramadol and its major metabolites by a validated HPLC assay with mass detection. FIG. 13 is a plot of the average tramadol plasma concentration (±SD) as a function of time for the sustained release formulation of the present invention in the fed (-▪-) and fasted state (-□-). As can be seen, plasma levels for the present invention in both the fed and fasted states formulation rise rapidly and remain relatively constant for at least 8 hours. The areas under the plasma tramadol concentration time curves are 220,879±27,047 ng·min/mL for the fed state and 189,313±82,551 ng·min/mL for the fasted state indicating substantially equivalent tramadol exposure in both states.

### Example 6

### Determination of the Drug Loading in the Particles of the Invention

The drug content of the beads was determined spectrophotometrically by crushing a weighed amount of coated or uncoated beads and dissolving the crushed beads in a known volume of water to give an acceptable absorbance reading. The UV spectrogram of tramadol HCl exhibits characteristic peaks at about 220 nm, 270 nm and 275 nm wavelengths. From the determined extinction coefficient for the drug at 270 nm, the concentration of drug in the sample was determined.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1) An rapid and extended release pharmaceutical composition:
a population of rapid release particles each comprising tramadol or a salt thereof and at least one excipient; and
a population of controlled release particles each comprising a core and a semipermeable coating enclosing the core, the core comprising tramadol or a salt thereof and at least one excipient, and the coating comprising a film-forming semipermeable polymer, a pore former and a plasticizer;
wherein the controlled release particles release tramadol or a salt thereof over an extended period of time in a substantially pH independent manner.

2) The composition of claim 1, wherein the amount of tramadol, or a salt thereof, present in the population of controlled release particles is greater than the amount of tramadol, or a salt thereof, present in the population of rapid release particles.

3) The composition of claim 2, wherein the population of rapid release particles comprises 25 to 100 mg of tramadol or a salt thereof.

4) The composition of claim 3, wherein the population of controlled release particles comprises 50 to 400 mg of tramadol or a salt thereof.

5) The composition of claim 1, wherein the amount of tramadol or a salt thereof present in the population of rapid release particles is 25% to 50% of the total amount of tramadol or a salt thereof present in the composition and the amount of tramadol or a salt thereof present in the population of controlled release particles is 75% to 50%, respectively, of the total amount of tramadol or a salt thereof present in the composition.

6) The composition of claim 1, wherein the rapid release particles release the tramadol or a salt thereof within one hour or less after exposure to an aqueous environment.

7) The composition of claim 6, wherein the controlled release particles release the tramadol or a salt thereof over a period of at least 8 to 12 hours after exposure to an aqueous environment.

8) The composition of claim 1, wherein the core of each of the controlled release particles comprises a rapid release particle formulation.

9) The composition of claim 1, wherein the film-forming semipermeable polymer comprises cellulose acetate butyrate, cellulose acetate propionate or a combination thereof.

10) The composition of claim 1, wherein the pore former is selected from the group consisting of sugar, polyol, water soluble or water erodible derivatized cellulose, and water soluble or water erodible polymer.

11) The composition of claim 1, wherein the plasticizer is selected from the group consisting of PEG having a molecular weight of 200 to 8000, ester of citric acid, ester of phthalic acid, and dibutyl sebacate.

12) The composition of claim 1, wherein the at least one excipient in the rapid release particles comprises a spheronization aid.

13) The composition of claim 12, wherein the spheronization aid comprises microcrystalline cellulose.

14) The composition of claim 13, wherein the at least one excipient in the rapid release particles comprises microcrystalline cellulose and sodium carboxymethylcellulose.

15) The composition of claim 13, wherein the at least one excipient in the rapid release particles comprises microcrystalline cellulose and starch.

16) The composition of claim 1, wherein the length or diameter of the rapid release particles ranges from 0.5 to 3 mm and the length or diameter of the controlled release particles ranges from 0.5 to 3 mm.

17) A dual release capsule dosage form comprising a capsule shell and a pharmaceutical composition, the pharmaceutical composition comprising:
a population of rapid release particles each comprising tramadol or a salt thereof and at least one excipient; and
a population of controlled release particles each comprising a core and a semipermeable coating enclosing the core, the core comprising tramadol or a salt thereof and at least one excipient, and the coating comprising a film-forming semipermeable polymer, a pore former and a plasticizer;
wherein the controlled release particles release tramadol or a salt thereof over an extended period of time in a substantially pH independent manner.

18) A controlled release particle that provides a substantially pH independent release of tramadol or a salt thereof, the particle comprising:
a core comprising tramadol or a salt thereof and at least one excipient; and
a semipermeable coating enclosing the core, the coating comprising a film-forming semipermeable polymer, a water soluble or water erodible pore former, and a plasticizer; wherein the pore former dissolves or erodes following exposure of the particle to an aqueous environment, thereby rendering the semipermeable membrane microporous so as to provide a pH independent release of tramadol from the core.

19) The particle of claim 18, wherein the tramadol or a salt thereof is released according to a first order, pseudo-first order, zero order, pseudo-zero order, or sigmoidal release profile.

20) A dosage form comprising:
a population of rapid release particles each comprising tramadol or a salt thereof and at least one excipient; and
a population of controlled release particles each comprising a core and a semipermeable coating enclosing the core, the core comprising tramadol or a salt thereof and at least one excipient, wherein the controlled release particles release tramadol or a salt thereof over an extended period of time in a substantially pH independent manner when exposed to an aqueous environment of use.

21) The dosage form of claim 20, wherein the dosage form is a solid oral dosage form.

22) The dosage form of claim 21, wherein the dosage form is selected from the group consisting of a capsule, caplet, tablet, sache, and particulate admixture.

23) The dosage form of claim 20, wherein the rapid release particles release tramadol in a substantially pH independent manner when exposed to an aqueous environment of use.

24) The dosage form or composition according to claim 1, wherein the immediate release particles release tramadol in a substantially pH independent manner when exposed to an aqueous environment of use.

25) The dosage form or composition of claim 24, wherein the immediate release particle completes release of tramadol within about 45 minutes after exposure to an aqueous environment of use.

26) A method of treating a disorder or symptom therapeutically responsive to tramadol or a salt thereof to a subject in need thereof comprising orally administering to the subject a pharmaceutical composition or a dosage form according to claim 1.

27) A method of administering tramadol to a subject comprising administering a unit dose of a dosage form or composition according to claim 1 to provide in the subject a plasma concentration of at least 100 ng of tramadol/ml of plasma for a period of at least eight hours following administration of the dosage form or composition to the subject.

28) The method of claim 27, wherein the plasma concentration ranges from 100 to 450 ng/ml.

29) The method of claim 28, wherein the period is at least twelve hours.

30) The method of claim 27, wherein the dosage form or composition comprises:
a population of rapid release particles each comprising tramadol or a salt thereof and at least one excipient; and
a population of controlled release particles each comprising a core and a semipermeable coating enclosing the core, the core comprising tramadol or a salt thereof and at least one excipient, wherein the controlled release particles release tramadol or a salt thereof over an extended period of time in a substantially pH independent manner.

31) The dosage form or composition of claim 1 further comprising an acidifying agent.

32) The dosage form or composition of claim 31, wherein the tramadol is present as the free base and the acidifying agent is adapted to form a salt with tramadol when the dosage form or composition is exposed to an aqueous environment.
